(19) United States
(12) Patent Application Publication
(10) Pub. No.: US 2014/0178895 A1
(43) Pub. Date: Jun. 26, 2014

(54) RECOMBINANT PROTEIN BIOSENSORS AND A METHOD FOR DETECTING THE PRESENCE OF AN ANALYTE MOLECULE

(71) Applicants: Saurabh Rajendra Nirantar, Singapore (SG); Farid John Ghadessy, Singapore (SG)

(72) Inventors: Saurabh Rajendra Nirantar, Singapore (SG); Farid John Ghadessy, Singapore (SG)

(21) Appl. No.: 14/006,961

(22) PCT Filed: Mar. 22, 2012
(86) PCT No.: PCT/SG2012/000098

§ 371 Date: Dec. 9, 2013

(30) Foreign Application Priority Data
Mar. 23, 2011 (SG) 201102087-2

(51) Int. Cl. G01N 33/58

(52) U.S. Cl. CPC G01N 33/581; G01N 33/582
USPC 435/7.9; 435/231; 435/188; 436/501; 530/350

(57) ABSTRACT

The present invention refers to a fusion protein biosensor, comprising a peptide or protein domain that binds an analyte of interest (A), an entity that can produce a detectable signal (B), and an entity that binds to B and modulate the signal produced by B when A is not bound to the analyte. A method and a kit for detecting a presence or amount of an analyte molecule using the fusion protein is also disclosed.

### CROSS-REFERENCE TO RELATED APPLICATION

This application makes reference to and claims the benefit of priority of a Singapore patent application for “Allosteric Modular Biosensors” filed on Mar. 23, 2011, and there duly assigned application number 201102087-2. The content of said application filed on Mar. 23, 2011 is incorporated herein by reference for all purposes, including an incorporation of any element or part of the description, claims or drawings not contained herein and referred to in Rule 20.5(a) of the PCT, pursuant to Rule 4.18 of the PCT.

### FIELD OF THE INVENTION

The present invention relates to recombinant protein biosensors and methods for detecting the presence of an analyte molecule, in particular, recombinant protein biosensors and methods for detecting the presence of an analyte molecule using analyte binding molecules.

### BACKGROUND OF THE INVENTION

One-step homogenous biosensors have the potential to significantly simplify and expedite analyte detection procedures as tedious washing steps or secondary detection reagents (like HRP labeled antibodies) that are the norm with current procedures such as ELISA are not required. However developing such biosensors has been a considerable undertaking, usually requiring starting from scratch for each and every analyte of interest. On the other hand, a wide variety of protein domains known to bind analytes of interest with high specificity and sensitivity exist, ranging from monoclonal antibodies and derivative single-chain variable fragments (scFvs) to artificial binding molecules based on scaffolds such as fibronectin to peptide aptamers.

Therefore, there remains a need to provide a modular and predictable mechanism to convert the above-mentioned binders into analyte responsive biosensors, which would greatly facilitate many laboratory procedures.

### SUMMARY OF THE INVENTION

In a first aspect, the invention relates to a fusion protein comprising a structure of

(Aₙ-B-Aₓ-C-Aₘ)_{y}

wherein
each A independently comprises a peptide or protein domain that binds an analyte of interest;
B comprises an entity that can produce a detectable signal;
C comprises an entity that, when A is not bound to the analyte of interest, binds to B and modulates the signal production by B;
n and m are each independently 0 or at least 1;
x is an integer of 0 or at least 1;
y is an integer of at least 1;
“-” represents a covalent bond or a linker comprising or consisting of one or more amino acids;
provided that if x is 0, n, m or both are at least 1.

In a second aspect, a method for detecting a presence or amount of an analyte molecule is provided. The method comprises contacting a fusion protein as defined in accordance to various embodiments of the present invention with the analyte molecule under conditions that allow binding of A to the analyte molecule; and detecting the presence of the analyte molecule by determining a signal produced by the fusion protein:analyte complex.

In a third aspect, a kit for detecting a presence of an analyte molecule or an amount of analyte is provided. The kit comprises a fusion protein in accordance to various embodiments of the present invention.

### BRIEF DESCRIPTION OF THE DRAWINGS

In the following description, various embodiments of the invention are described with reference to the following drawings, in which:

FIG. 1 shows (a) a schematic depiction of a biosensor comprising the fusion protein Tem1 lactamase-HIV p17 epitope (EKIRLR)-beta lactamase inhibitor protein (BLIP); and (b) the rate of substrate turnover, as measured by nitrocefin absorbance of the HIV p17 epitope biosensor in the presence of various analytes, in accordance to various embodiments;

FIG. 2 shows OD492 increase in the presence of various analytes for the biosensor mentioned in FIG. 1, in accordance to various embodiments;

FIG. 3 shows the response of DO1 biosensor (as in FIG. 1, but with the peptide epitope of DO1 antibody between Tem1 and BLIP instead of EKIRLR peptide) monitored by nitrocefin cleavage absorbance at OD 492 nm, in accordance to various embodiments;

FIG. 4 shows substrate turnover for various analytes being added to DO1 biosensor, in accordance to various embodiments;

FIG. 5 shows a schematic representation of the rigidification of Mdm2 upon binding its cognate analytes, in accordance to various embodiments;

FIG. 6 shows the response to Mdm2 biosensor based on triplicates of various analytes, in accordance to various embodiments;

FIG. 7 shows images of visual appearance of the reaction of FIG. 6, in accordance to various embodiments;

FIG. 8 shows the response to Mdm2 biosensor, in accordance to various embodiments;

FIG. 9 shows images for JM109 cells being transformed with expression plasmids encoding indicated biosensor (Kan resistance cassette) and DO1 ScFv (Chlor resistance cassette), in accordance to various embodiments;

FIG. 10 shows a schematic representation of the coiled-coil binding for fluorophore-quencher configuration, in accordance to various embodiments;

FIG. 11 shows the response of p53 short peptide against specific DO1 and non-specific (bp53) antibodies, in accordance to various embodiments; and

FIG. 12 shows images of samples of FIG. 11 excited by fluorescence, in accordance to various embodiments.

### DETAILED DESCRIPTION OF THE INVENTION

The following detailed description refers to, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not necessarily mutually exclusive, as some embodiments can be combined with one or more other embodiments to form new embodiments.

In a first aspect, a fusion protein comprising a structure of

(Aₙ-B-Aₓ-C-Aₘ)_{y}

wherein each A independently comprises a peptide or protein domain that binds an analyte of interest; B comprises an entity that can produce a detectable signal; C comprises an entity that, when A is not bound to the analyte of interest, binds to B and modulates the signal production by B; n and m are each independently 0 or at least 1; x is an integer of 0 or at least 1; y is an integer of at least 1.

The symbol “-”represents a covalent bond or a linker comprising or consisting of one or more amino acids.

In the context of various embodiments, the term “fusion” or “fusion protein” refers to a covalent linkage of two or more proteins or fragments thereof via the respective peptide backbones. The linkage may also be co-linear or co-translational. A fusion protein is an artificial protein or polypeptide derived from fusing the two or more proteins or fragments thereof. In some examples, the proteins and fragments thereof may be from different sources.

As used herein, the term “domain” with reference to a protein may refer to an independently folding peptide structure that may naturally be part of a larger protein. For example, a domain in the sense of the present invention may include one or more amino acid stretches that have a secondary, optionally a tertiary and optionally a quaternary structure and fold independently from other parts of the protein that may not be present in the isolated domain.

The term “structure” refers to a chemical structure.

The term “binds” refers to an interaction between a target and a potential binding component wherein the potential binding component preferentially associates with the target. The association may be measured by a level. The term “binding component” refers to a compound that has a statistically significant association with a target molecule. The binding may generally be specific or non-specific. In various embodiments, the binding of the protein or peptide domains may be specific to the analyte of interest.

The term “entity” may refer to but is not limited to a chemical moiety, such as a peptide, or a protein, or a protein domain, or organic molecule. The entity may comprise or consist of a chromophore or fluorophore a quenching group, wherein this quenching group can absorb emission (or excitation) energy from a fluorophore or chromophore, usually in spatial proximity, and thus quench the signal generated by the fluorophore or chromophore.

As used herein, the term “produce” may interchangeably be referred to generate, send, give off, give or emit. The term “detectable signal” refers to a signal that can be detected or measured directly or indirectly. For example, the detectable signal may be detectable or measurable by physical, spectroscopic, photochemical, biochemical, immunochemical or chemical means. The detectable signal may be produced directly or indirectly by reaction or interaction with a suitable conjugate, for example, a substrate. The detectable signal may be an “indicator molecule”.

In the context of various embodiments, the term “modulates” refers to change. The change may include stimulation. For example, the signal may be modulated by an increase in the signal or a decrease in the signal.

The term “linker” refers to a linking component or a spacing moiety which can covalently or non-covalently link a compound to a solid support or a protein domain or another chemical entity. Linkers may be selected based on their length, chemical stability, affinity, flexibility.

In various embodiments, the linker comprises or consists of one or more amino acids. The term “amino acid” refers to naturally occurring and artificially produced amino acids, and also amino acid analogs and amino acid mimetics that function in a similar manner to the naturally occurring amino acids. Amino acid analogs refer to compounds that have the same basic chemical structure as the naturally occurring amino acids.

In one example, the one or more amino acids may refer but are not limited to one amino acid, two amino acids, three amino acids, four amino acids, five amino acids, six amino acids, seven amino acids, eight amino acids, nine amino acids, ten amino acids, or twenty amino acids.

The use of chemical linkers may be, for example, for use to connect the fluorophore/quencher to the conditionally fluorescent peptides (eg. as shown in FIG. 10)

In various embodiments, B and C each may independently comprise a peptide or protein domain. For example, B may be a peptide or protein domain coupled to a substance that can produce the detectable signal.

In one embodiment, B may be an enzyme or may have enzymatic activity. The enzyme may be a catalytic peptide. The enzyme may be capable of providing a convenient read-out. For example, the enzyme may be but is not limited to lactases, catalases, amylases, beta-lactamases, cephalosporinases, penicillinases, cephalosporinases, and carbenicilliniases. In various other embodiments, B may be beta-galactosidase or alkaline phosphatase.

In such embodiments, C may be an inhibitor of the enzyme, for example a protein inhibitor. The inhibitor may be a competitive inhibitor or an allosteric inhibitor. Binding of the inhibitor to the enzyme is reversible. For example, the protein inhibitor may be but is not limited to a protein inhibitor such as alpha 1-antitrypsin, C1-inhibitor, antithrombin, alpha 1-antichymotrypsin, plasminogen activator inhibitor-1, neuroserpin, or a beta-lactamase inhibitor protein (BLIP), sulbactam, and tazobactam.

In various embodiments, the length of the linker between the peptides or protein domains may be selected such that it allows the interaction of B and C when A is not bound to the analyte of interest, and it prevents the interaction of B and C when A is bound to the analyte of interest. The analyte of interest interchangeably refers to an analyte under test.

In one embodiment, the binding of B and C may be impaired when all A moieties of the fusion protein are bound to an analyte, optionally the same analyte.

In specific embodiments, the fusion protein may comprise the structure of (B-A-C)_{y}. The structure is obtained when m and n are each independently 0; and x is 1.

In other embodiments, B may comprise a chromophore or fluorophore and C may comprise a substance that absorbs emission energy of the chromophore or fluorophore (quencher) or vice versa.

In one embodiment, B is a chromophore or fluorophore. In another embodiment, B may be conjugated to the chromophore or fluorophore with B being a peptide, protein or protein domain. B may also be a fluorescent protein or peptide.

In one embodiment, C is a substance that absorbs emission energy of the chromophore or fluorophore. In another embodiment, C may be conjugated to such a substance that absorbs emission energy of a chromophore or fluorophore. In still another embodiment, C may be fluorescent peptide or protein, for example a conditionally fluorescent peptide. As used herein, the term “conjugated” means to be joined together, to be coupled, or to act or operate as if joined. Usually, conjugation occurs by covalent linkage or ionic interaction.

In one embodiment, B or C may comprise or be a fluorophore and the other is or comprises a substance that absorbs emission energy of the fluorophore, i.e.,e a quencher. In an embodiment, B may comprise a fluorophore and the substance that absorbs emission energy of the chromophore (or fluorophore) may be a quencher.

For example, the fluorophore may be but is not limited to fluorescein, 5-((2-Aminoethyl)amino)naphthalene-1-sulfonic acid (EDANS), isothiocyanate, coumarin, cyanine and rhodamine. Examples of a quencher may be but not limited to dark quencher, dimethylaminoazosulfonic acid, black hole quenchers, dabcyl, and Q×1 quencher. More specifically, dabcyl may be a quencher to inhibit fluorophores in the context of conditionally fluorescent peptides (for example, as illustrated in FIG. 10).

In other embodiments, the fluorophore may be green fluorescent protein (GFP). If GFP or derivatives thereofare used as a read-out molecule, then another fluorescent protein such as but not limited to red fluorescent protein (RFP) may be used as the interaction partner. In such a case, the detection makes use of the so-called fluorescence resonance energy transfer (FRET) technique, where the one fluorescent moiety, when in close proximity to the other (in the absence of analyte), will absorb photons emitted by the other fluorophore via Fluorescence Resonance Energy Transfer (FRET) and re-emit a longer wavelength photon. In case of using GFP and RFP, for example, the GFP emission photons are not seen when RFP is in close proximity; as RFP is inhibiting GFP emission so that only RFP emission is detected. When analyte is added and RFP is moved away from GFP more of the GFP emission photons will be detected and a GFP signal is detectable. In this way, a FRET acceptor such as RFP may also be considered an inhibitor of a FRET donor (such as GFP). However, in preferred embodiments of the invention not both, B and C, comprise or are fluorophores and the detection principle is not based on FRET.

In various embodiments, when more than one A is present in the fusion protein, each A may be the same, or two or more A may be different. In one example, the two or more A may be arranged to bind to the same analyte.

In various embodiments, A may comprise an antibody, antibody fragment, peptide aptamer, peptide antigen, or peptide antigen fragment.

As used herein, the term “antibody” may be used in the broadest sense and covers polyclonal antibodies, monoclonal antibodies, multispecific antibodies, single domain antibodies, and phage antibodies. Antibodies may refer to fragments of antibodies. The term “antibody fragment” means a portion of the full length antibody, generally the antigen binding or variable region thereof. For example, an antibody fragment may include single chain antibodies (scFv) or binding fragment (Fab). Antibodies may be interchangeably referred to as immunoglobulin. Varieties of antibodies may be, for example, IgA, IgD, IgE, IgG and IgM. Examples of antibodies may be but not limited to anti-HIV p17 epitope, p53 (DO-1) monoclonal antibody, and anti c-myc antibody.

In some examples, A may comprise naturally occurring ligands or interacting partners. For example, Mdm2 may be used as A for the detection of p53.

The “peptide aptamer” may be a combinatorial protein reagent that binds to target proteins with a high specificity and a strong affinity. For example, the peptide aptamer may inhibit the function of a protein in vivo.

The term “antigen” generally refers to a molecule capable of being bound by an antibody. For example, an antigen may be but is not limited to pathogen derived proteins/molecules, molecules of medical interest such as insulin, hcG, etc. In one embodiment, the antigen or antigen fragment may comprise or consist of an antigenic determinant or epitope.

In some embodiments, x may be the integer 1 or 2. In other embodiments, x may be an integer of more than 2. For example, when x=1, the fusion protein may comprise the structure of (Aₙ-B-A-C-Aₘ)_{y}. When x=2, the fusion protein may comprise the structure of (Aₙ-B-A-A-C-Aₘ)_{y}. The structure of the fusion protein is given in N-terminal to C-terminal orientation.

The term “C-terminal” or “C-terminus” used herein is not equivalent to the symbol “C” in the structure of the fusion protein.

In other embodiments, the respective positions of B and C may be exchanged within the fusion protein such that the fusion protein comprises a structure of (Aₙ-C-Aₓ-B-Aₘ)_{y}.

In various embodiments, the binding of C to B may decrease or inhibit signal production by B.

In other embodiments, C may initiate or increase signal production by B upon binding. Such initiation or increase may be upon the binding of B and C. As such, C may be or may involve an activator or an enhancer. For example, an activator may be an enzyme activator, which is a molecule that binds to an enzyme and increases its activity. For illustrative purposes, an example of an enzyme activator may be the α fragment of beta-galactosidase and variants thereof, which activates the Ω fragment. Other enzyme activators may work in a similar manner. For example, an enhancer may be an enzyme enhancer, which can bind to a non-active site and cause a conformation change which enhances enzyme function.

In one embodiment, B may be beta lactamase or homologs, fragments and variants thereof, wherein the homology, fragments and variants at least partially retain enzymatic activity. In other embodiments, B may be various beta lactamases or homologs, fragments and variants thereof, wherein the homology, fragments and variants at least partially retain enzymatic activity. In one specific embodiment, B is the beta lactamase TEM1 or variants thereof. “Variant”, as used herein, refers to a protein that differs from a consensus sequence or the accepted wildtype sequence by at least one amino acid variation. The variant may be but is not limited to a natural variant, or a M69L variant, or a E104K variant. The beta lactamase TEM1 may comprise the amino acid sequence set forth in SEQ ID NO: 1 (UniProtKB accession number Q5QJI7). In one specific embodiment, the beta lactamase TEM1 may comprise a homolog or fragment or variant of the amino acid sequence set forth in SEQ ID NO: 1.

“Homologs”, as used herein, refer to two proteins that have similar amino acid sequence. Homologs include orthologs, or paralogs. “Fragments”, as used herein, refer to a portion of amino acid sequence, that is, a polypeptide comprising fewer than all of the amino acid residues of the protein.

Generally, as used herein, the term “beta lactamase” includes multiple beta lactamases, for example, any of Class A beta lactamases, Class B beta lactamases, Class C beta lactamases, and/or Class D beta lactamases. In one embodiment, the beta lactamase is a Class A beta lactamase, such as, for example, TEM1.

In another embodiment, C may be beta lactamase inhibitor protein (BLIP) or BLIP-I or BLIP-II or fragments, homologs and variants thereof that retain at least partially the binding activity for beta lactamase. In one specific embodiment, C is a beta lactamase inhibitor protein (BLIP) or variants thereof. The terms “variant”, “homologs” and “fragments” are as defined above. The BLIP may have the amino acid sequence set forth in SEQ ID NO: 2 (UniProtKB accession number Q18BP3). In one specific embodiment, the BLIP may comprise a homolog or fragment or variant of the amino acid sequence set forth in SEQ ID NO: 2. In an embodiment, C may comprise molecules with similar function such as BLIP-I and BLIP-II.

In various embodiments, A may be an antigenic peptide. The antigenic peptide may comprise or may consist of an amino acid sequence selected from the group consisting of those set forth in SEQ ID NOs: 3, 8-10, 12-13.

In one embodiment, the symbol “-” may represent a linker comprising more than one amino acids and wherein the linker peptide forms a coiled coil structure.

As used herein, the term “coiled coil” may refer to a peptide or protein sequence usually with a contiguous pattern of hydrophobic residues spaced 3 or 4 residues apart and assembles (or folds) to form a multi-meric bundle of helices. A pair of coiled coils may be brought toward each other to form a parallel configuration or an anti-parallel pair configuration. At a same end of the parallel configuration, the coiled coils may not touch each other but the coiled coils may be close enough to form a reaction thereof. In this regard, use of anti-parallel pairs may be considered to be more suitable than parallel pairs.

In various embodiments, the fusion protein may have the amino acid sequence set forth in any one of SEQ ID NOs: 4-5, 11, 14-15.

In a second aspect, a method for detecting a presence or amount of an analyte molecule is provided. The method comprises contacting a fusion protein according to various embodiments of the present invention with the analyte molecule under conditions that allow binding of A to the analyte molecule; and detecting the presence of the analyte molecule by determining a signal produced by the fusion protein:analyte complex.

As used herein, the term “contacting” may refer to reacting or binding. Contacting may be bringing a compound and a target together such that the compound can affect the activity of the target. Contacting may be but is not limited to being performed in a test tube or a petri-dish. Contacting may involve incubation.

The term “detecting” refers to monitoring, determining, or sensing. The term “presence” may refer to the existence or a measureable level of.

The term “under conditions” refers to being subject to a certain set of requirements or parametric control to achieve binding of A to the analyte molecule. For example, the conditions may be but is not limited to temperature and/or length of time. The term “determining” refers to measuring, deriving, or checking. The term “fusion protein:analyte complex” is formed when the fusion protein binds with the analyte, thus forming a complex. As used herein, an “amount” may represent a measurable level.

The terms “fusion protein”, “detectable signal”, and “binding” are as defined hereinabove.

In various embodiments, the presence or amount of the analyte molecule may be detected in a sample.

In another embodiment, the step of contacting the fusion protein with the analyte molecule may comprise contacting the fusion protein with a sample suspected to contain the analyte molecule.

For example, the step of detecting the presence of the analyte molecule may further include comparing the detected analyte molecule with a control measurement. The “control” measurement may be a positive or negative control measurement. The control measurement serves as a reference or basis against which comparison may be made to the detected measurement.

In various embodiments, the signal produced by the fusion protein may be determined by fluorescence, absorbance, luminescence, enzymatic activity, use of a detectable functional product, use of a selectable phenotype, use of a screenable phenotype that produces an activity due to a phenotypic change. For example, the selectable phenotype may comprise an antibiotic resistance. In an example, the signal produced by the fusion protein may be determined by use of in vivo expression.

The B-C binding and the A-analyte molecule binding are mutually incompatible. In other words, the B-C binding cannot bind to the A-analyte molecule binding.

In various embodiments, the method may be performed in a living cell in vivo or in vitro (ex vivo). The cell may be used for a drug screening assay. For example, the use of the biosensor may be for directed evolution studies in cells (typically bacteria).

In a third aspect, a kit for detecting a presence of an analyte molecule or an amount of analyte is provided. The kit comprises a fusion protein as defined above.

The kit may further comprise a substance for detecting the signal produced by the fusion protein is provided. The fusion protein may be as defined hereinabove.

In order that the invention may be readily understood and put into practical effect, particular embodiments will now be described by way of examples and not limitations, and with reference to the figures.

### EXAMPLES

### Example 1

Anti c-myc Epitope Biosensor

Example 1 describes the development of an anti c-myc epitope biosensor. Apart from a high affinity binding protein, an enzyme capable of providing a convenient read-out (beta-lactamase TEM1 (SEQ ID NO: 1) in this example, but it is noted that any other enzyme may be used) and a protein inhibitor of the said enzyme (beta-lactamase inhibitor protein, or BLIP (SEQ ID NO: 2), in this case) are required. In Example 1, the anti c-myc epitope biosensor was constructed by placing the linker encompassing the c-myc epitope GGSEQKLISEEDGG (SEQ ID NO: 3) between BLIP and TEM1. The anti c-myc epitope biosensor may be BLIP-GGGS-c myc epitope-GGGGSGGGGSGGGGSGGGGG-TEM1 or TEM1-GGGS-c myc epitope-GGGGSGGGGSGGGGSGGGGG-BLIP (SEQ ID NOs: 4 and 5) wherein -GGGS- (SEQ ID NO: 6) and -GGGGSGGGGSGGGGSGGGGG- (SEQ ID NO: 7) are examples of linkers.

The rationale is that while in the absence of anti c-myc antibody the c-myc epitope peptide would be flexible, allowing BLIP-TEM1 binding, the introduction of anti c-myc antibody would rigidify the c-myc epitope and abolish the flexibility required to allow BLIP-TEM1 binding, thereby activating TEM1.

As used herein, the term “rigidify” refers to being rigid, in contrast, to being flexible. The level of rigidness or hardness may be measurable. In this context, rigidness may occur at the backbone of the peptide or protein domain.

The analyte responsiveness of the anti c-myc biosensors reveals strong support for the idea that integrating a read-out providing module such as TEM1, its inhibitor (BLIP) and analyte binding module(s) in such a manner that BLIP-TEM1 binding and binder-analyte binding are mutually incompatible is a viable generic method of generating homogenous one-step biosensors solely using pre-existing well-characterized protein domains. At the same time, the sensitivity obtained with the current first generation biosensors is low. Solutions to improve the sensitivity include placing an analyte binding module outside the BLIP-TEM1 interaction to increase the local concentration of the analyte, which may then subsequently be bound by a second binding module whose binding is incompatible with TEM1-BLIP interaction. The BLIP-TEM1 affinity may be fine-tuned using known mutants of BLIP to maximize signal to noise, lengthening the linker to decrease the background, using high signal fluorescent or chemiluminescent substrates instead of low signal chromogenic substrates and secondary amplification strategies. Additionally, in silico modeling needs to be explored to narrow down the search for the most promising configurations, linker lengths etc.

### Example 2

Detection of anti-HIV p17 Epitope Antibody

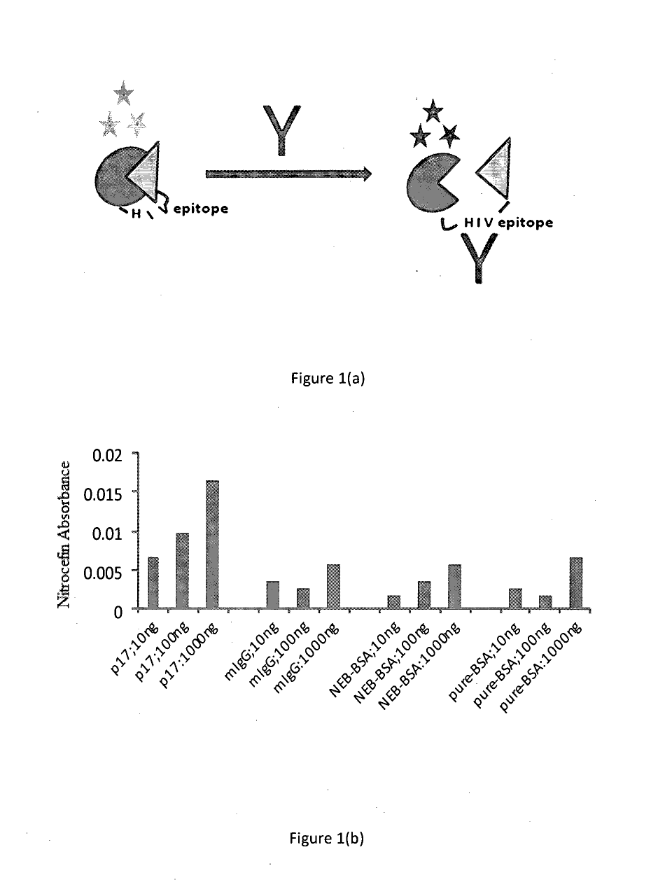

The conserved HIV p17 epitope EKIRLR (SEQ ID NO: 8) was placed as a linker joining the C terminus of TEM1 and the N terminus of BLIP (FIG. 1(a)). It was expected that binding of the relevant antibody (mouse monoclonal anti-p 17, Clone 32/1.24.89, Zeptometrix) to this epitope would rigidify the epitope and constrain the flexibility required to allow easy Tem1-BLIP binding. FIG. 1(b) shows the rate of substrate turnover, as measured by nitrocefin absorbance (y-axis) of the HIV p17 epitope biosensor in the presence of various analytes.

In FIG. 1(b), starting from the left, p17 refers to the anti-EKIRLR mouse monoclonal antibody. The amount of this antibody present in the 25 μl reaction is mentioned (10, 100 or 1000 ng). The next 3 histograms show biosensor response to various amounts of mouse whole IgG (mIgG, used as a negative control). The next two sets of three refer to BSA, as a negative control, from different sources, also used at 10, 100 or 1000 ng in a 25 μl reaction. The gradients were normalized by substracting the rate of nitrocefin turnover in the absence of any analyte (background).

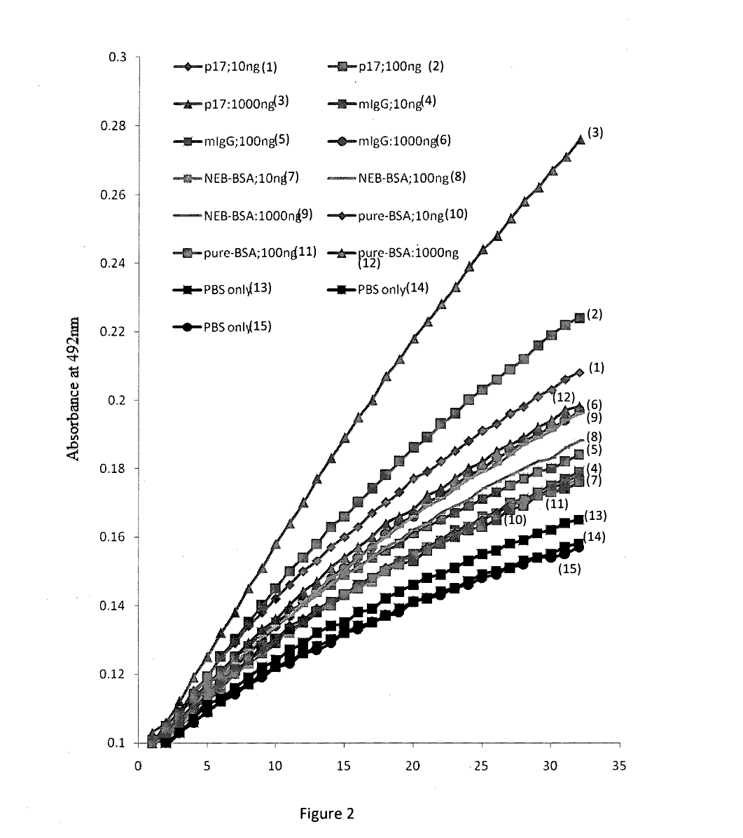

FIG. 2 shows OD492 (used to estimate nitrocefin cleavage by TEM1 lactamase, y-axis, read number on x-axis) increase in the presence of various analytes. As in FIG. 1, p17 refers to anti-HIV p17 mouse monoclonal antibody, mIgG refers to mouse whole IgG used as a negative control, NEB-BSA and pure-BSA refer to BSA from two different sources used as negative controls, PBS refers to background biosensor activity in buffer in the absence of any analyte. The amounts after the semi-colons refer to the amounts of the analytes present a 25 μl reaction.

Detection of DO1 Antibody, and its Use in a Competitive Assay to Detect Various Amounts of a p53 Derived Peptide

The DO1 antibody binds to the SDLWKLL (SEQ ID NO: 9) linear peptide epitope from p53. This amino acid sequence was placed between TEM1 and BLIP similar to the anti-HIV peptide mentioned above. The biosensor was expressed and tested for responsiveness to DO1 antibody. Detection of free p53 derived peptide in solution was demonstrated by titrating in various amounts of free p53 peptide into the reaction containing DO1 antibody and biosensor. It was expected that the degree of activation of biosensor would drop as the antibody epitope binding sites became blocked by free p53-derived peptide, and the amount of p53 peptide present may be estimated by the extent of the drop in activation. This concept may be extrapolated to free proteins in solution or cell lysates, epitope tags such as myc or HA etc.

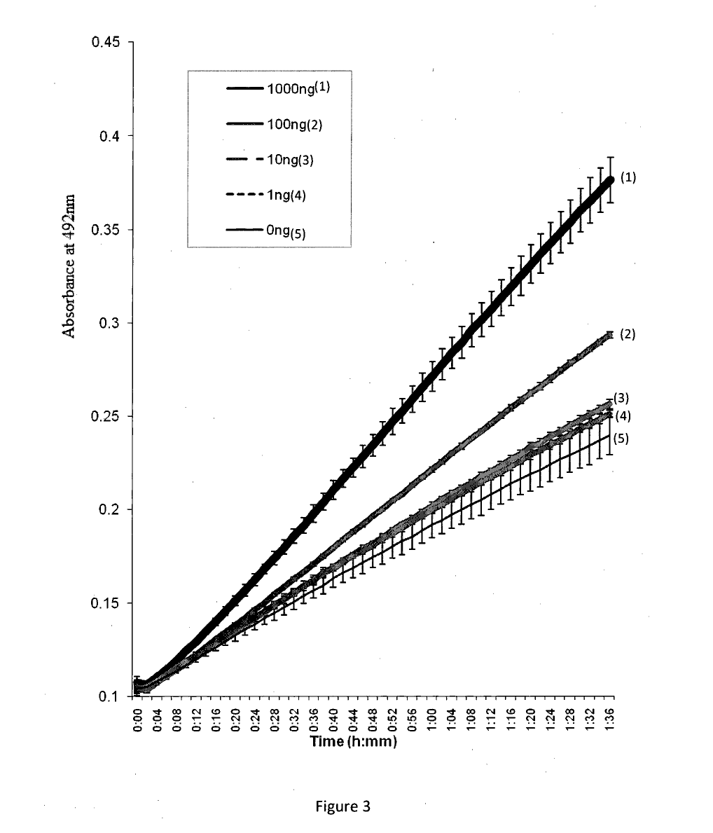

FIG. 3 shows increasing amounts of DO1 antibody (0, 1, 10, 100 and 1000 ng) were added to biosensor in a 25 μl reaction, and the response monitored by nitrocefin cleavage absorbance at OD 492 nm.

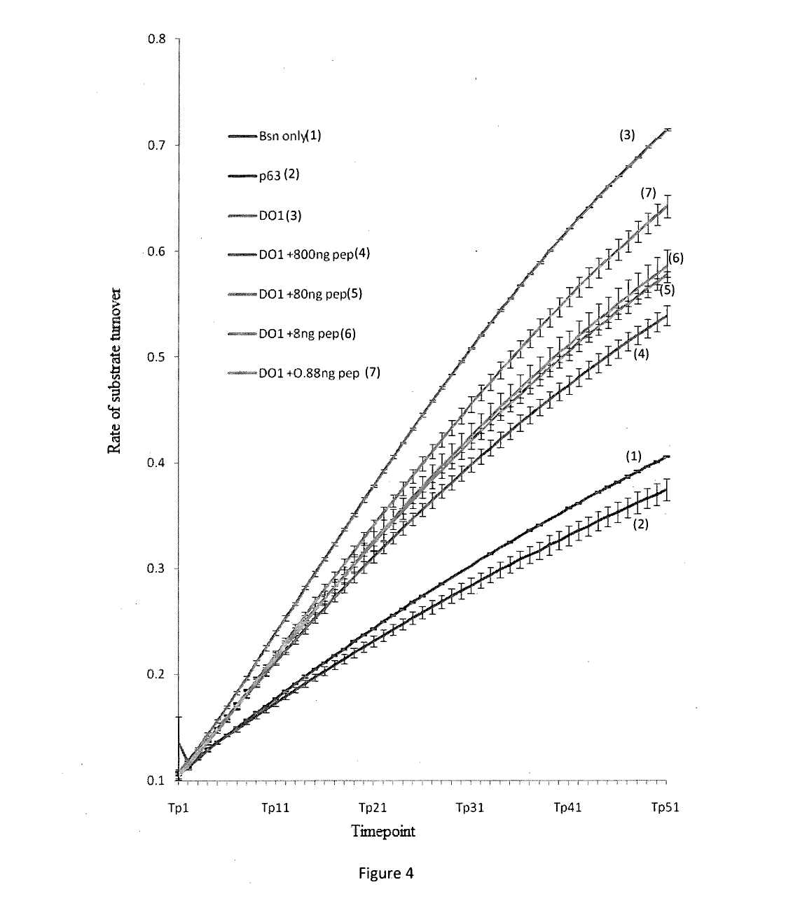

FIG. 4 shows various analytes were added to DO1 biosensor and the rate of substrate turnover was monitored (timepoints on x-axis). 1 μg of p63 antibody (negative control) and DO1 antibody (positive control are shown) along with buffer only control. Various amounts of free DO1 epitope peptide were added into the reaction to demonstrate competitive inhibition of biosensor due to occupation of the epitope binding sites in DO1.

### Example 3

Detection of Mdm2 Ligands Nutlin and p53 Derived Peptide Using a Mdm2 Biosensor

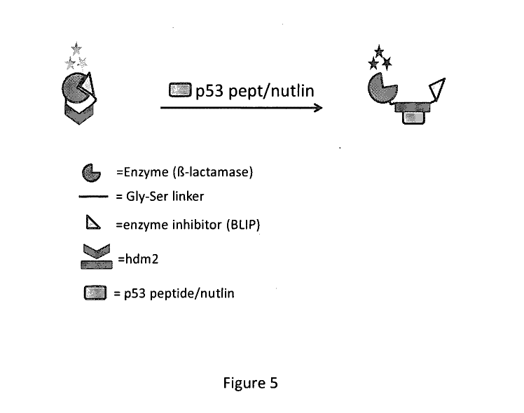

The N terminus of Mdm2 binds and inhibits p53 and is an attractive target for cancer therapy. It is bound by a small molecule, nutlin which is a competitive inhibitor of p53 binding to Mdm2 (SEQ ID NO: 10; UniProtKB accession number Q00987). In addition it is bound by a linear peptide derived from p53. A Mdm2 based sensor was constructed by placing the Mdm2 N terminus between TEM1 and BLIP. It was expected that the Mdm2 N terminus, which is known to have a molten globule structure would be distorted due to the constraint of spanning the distance from the C terminus of BLIP to the N terminus of TEM1. Addition of interactants like nutlin or p53 derived peptide may rigidify the Mdm2 N terminus and drive it to adopt its compact structure, leaving it unable to span the Tem1-BLIP distance, thereby separating some of the TEM1 molecules from BLIP, resulting in increased activity. This concept is schematically illustrated in FIG. 5.

In FIG. 5, it is shown how rigidification of Mdm2 upon binding its cognate analytes may lead to increased TEM1 lactamase activity.

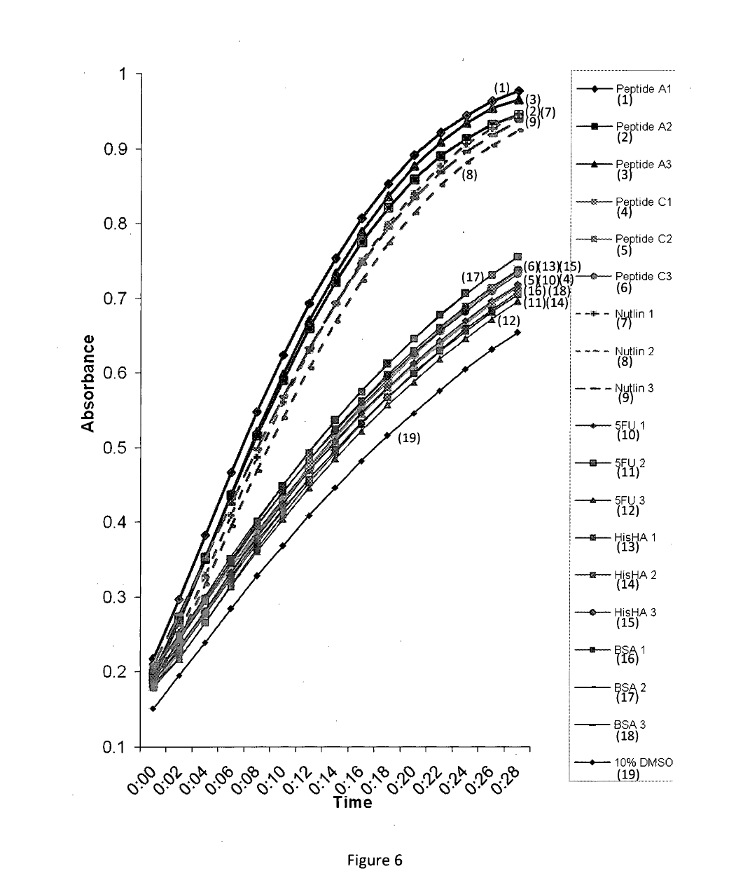

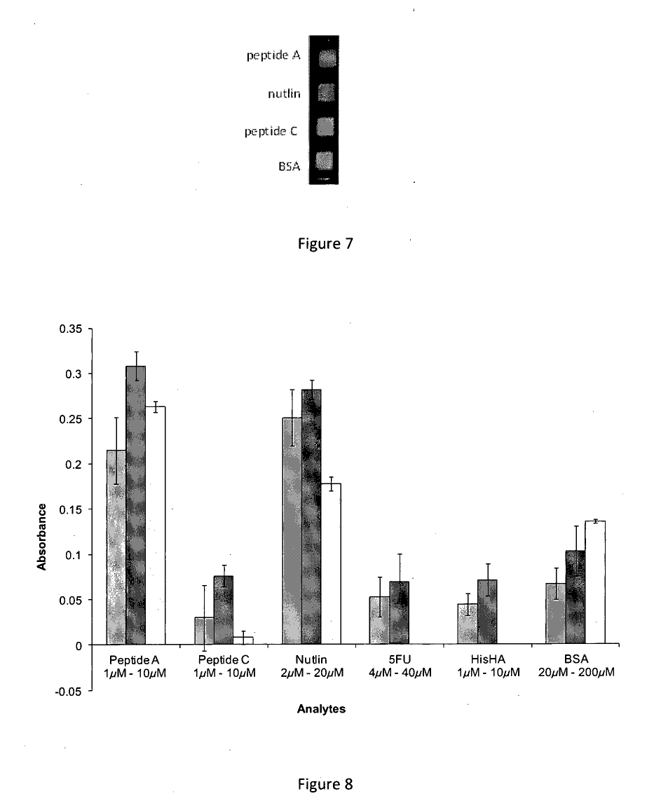

FIG. 6 shows the findings wherein various analytes in triplicates (indicated by the suffixed numbers 1-3) were added to Mdm2 biosensor in a 25 μl reaction. The final concentration of each analyte is 4 μg/ml. 1 μl of 10% DMSO (solvent used for the peptides and nutlin) was added as a buffer only control. The known interactants nutlin and peptide A (p53 derived Mdm2 binding peptide) show higher rates of substrate turnover than the negative controls peptide C (mutated p53 peptide), HisHA (non-interacting peptide), 5 FU (used as a small molecule control), BSA and no-analyte control. The visual appearance of the reactions is shown in FIG. 7.

FIG. 8 shows various concentrations of indicated interacting and non-interacting analytes were added to Mdm2 biosensor. The rates of substrate turnover in response as plotted. BSA is at a much lower concentration than the other analytes as its molecular weight is much greater. The mass/volume concentration of BSA is comparable to the other analytes.

### Example 4

Biosensor Function in In Vivo Assay

The DO1 biosensor described in FIGS. 3-4 (Tem1-SDLWKLL-BLIP) (SEQ ID NO: 11) was expressed in the periplasm of E. coli. Two derivatives with the wild type (WT) SDLWKLL sequence mutated to SGLWKLL and AGLWKLL as denoted in SEQ ID NOs: 12 and 13 respectively were expressed as controls. DO1 scFv was also expressed in these cells and directed to the periplasm using a suitable signal peptide. It was expected that DO1 scFv would bind to and activate the DO1 biosensor. As TEM1 activation leads to resistance to ampicillin and related antibiotics, it was expected a growth advantage would be conferred on the bacteria expressing a biosensor with the correct (SDLWKLL) but not the mutant (SGLWKLL and AGLWKLL) epitope between TEM1 and BLIP (SEQ ID NOs: 14 and 15, respectively). This method may then be used in screening for desirable properties in DO1 scFv, such as binding to a novel epitope, for instance.

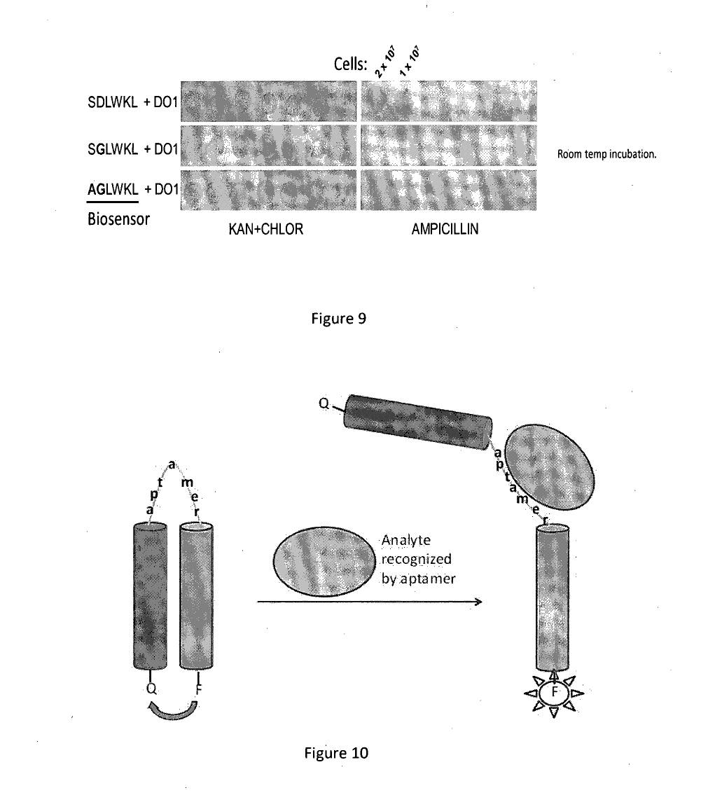

FIG. 9 shows JM109 cells transformed with expression plasmids encoding indicated biosensor (Kan resistance cassette) and DO1 ScFv (Chlor resistance cassette). Cells were grown to OD ˜0.5 and plated on ampicillin plates (right panel) or Kan+Chlor plates (left panel). Interaction between DO1 antibody and biosensor with correct epitope (SDLWKL) gives rise to lactamase activity and survival on ampicillin plates (right panel, top row). Reduced or no growth is seen when the epitope is mutated in the SGLWKL and AGLWKL biosensors (right panel, second and third row). Left panel indicates equivalent cell numbers used in assay.

### Example 5

Conditionally Fluorescent Peptide Biosensor

The biosensor concept described herein does not have to be limited to an enzyme-inhibitor system. The concept was extrapolated to a fluorophore-quencher pair. The fluorophore EDANS and the quencher dabcyl were used. As the fluorophore-quencher do not have significant intrinstic affinity for each other, the fluorophore and quencher were fused each to one member of a coiled-coil pair known to bind each other with high affinity in an antiparallel orientation at positions that place the fluorophore and quencher in close proximity, enabling efficient quenching. The N and C termini of the coils were linked by a peptide recognized by the DO1 antibody, analogous to the usage of this same peptide to link TEM1-BLIP N and C termini in the DO1 biosensor (see Example 2 above). The constraints imposed by having to link N and C termini of the coils should cause the DO1 epitope peptide to adopt a loop orientation, which, as with the enzymatic biosensors, should be disrupted by the rigidification engendered by cognate antibody binding. This may strain the coiled-coil binding, and cause a percentage of the coiled coils to become unbound, increasing fluorophore-quencher distance, enabling fluorescent emission from the fluorophore. This concept is schematically depicted in FIG. 10.

FIG. 10 shows that in the absence of analyte, the aptamer adopts a flexible loop conformation required to allow coiled-coil binding, thereby keeping the fluorophore close to the quencher. Analyte binding compels the aptamer to adopt a different conformation, straining the coiled coils, leading to their separation and therefore increased fluorescence.

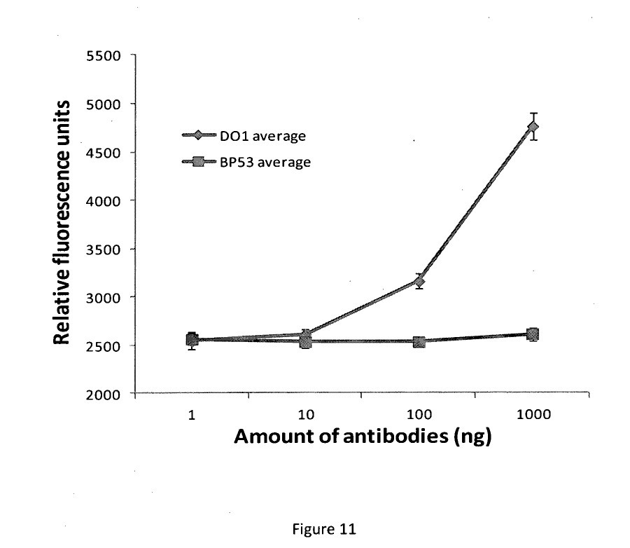

The peptide described above was synthesized chemically and tested. FIG. 11 shows the response of the conditionally fluorescent p53 short peptide (with the DO1 antigen peptide SDLWKLL between the antiparallel coiled coils) against specific (DO1) and non-specific (BP53) antibodies. In FIG. 11, the peptide with a p53 derived DO1 epitope linking the N and C termini of coiled coils carrying a fluorophore and quencher as in FIG. 10 were tested with cognate (DO1) and non-cognate (BP53) antibodies in a 25 μl reaction. The amount of antibody is each reaction is shown on the x-axis. Each data point represents triplicate experiments. DO1 antibody clearly leads to significantly higher fluorescence, as predicted.

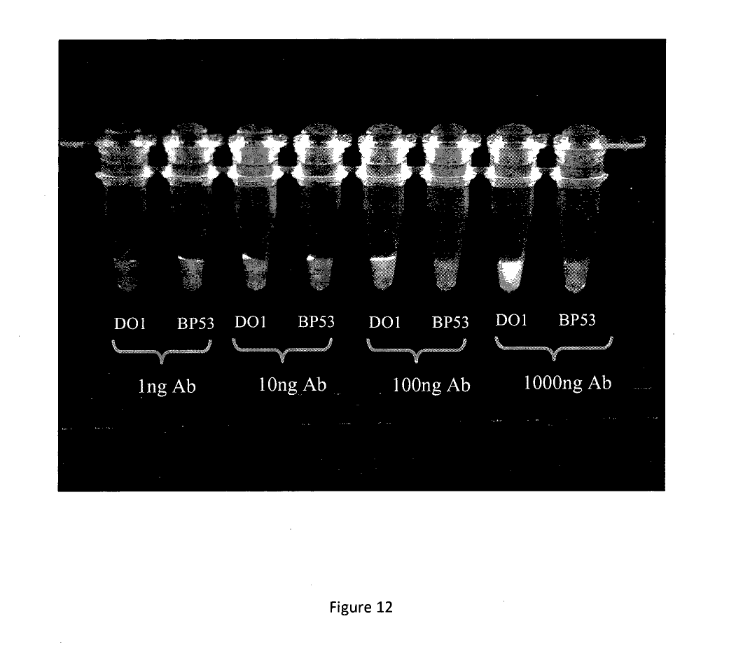

FIG. 12 shows samples from FIG. 10 being excited and the resulting emission was imaged.

The listing or discussion of a previously published document in this specification should not necessarily be taken as an acknowledgement that the document is part of the state of the art or is common general knowledge. All documents listed are hereby incorporated herein by reference in their entirety.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognise that the invention is also thereby described in terms of any individual member or subgroup of members of the Markush group.

1. A fusion protein comprising a structure of
(Aₙ-B-Aₓ-C-Aₘ)_{y}
wherein
each A independently comprises a peptide or protein domain that binds an analyte of interest;
B comprises an entity that can produce a detectable signal;
C comprises an entity that, when A is not bound to the analyte of interest, binds to B and modulates the signal production by B;
n and m are each independently 0 or at least 1;
x is an integer of 0 or at least 1;
y is an integer of at least 1;
“-” represents a covalent bond or a linker comprising or consisting of one or more amino acids;
provided that if x is 0, n, m or both are at least 1.

2. The fusion protein of claim 1, wherein B and C each independently comprises a peptide or protein domain.

3. The fusion protein of claim 1, wherein B is a peptide or protein domain coupled to a substance that can produce the detectable signal.

4. (canceled)

5. The fusion protein of claim 2, wherein the length of the linker between the peptides or protein domains is selected such that it allows the interaction of B and C when A is not bound to the analyte of interest, and it prevents the interaction of B and C when A is bound to the analyte of interest wherein preferably, the binding of B and C is impaired when all A moieties of the fusion protein are bound to an analyte, optionally the same analyte.

6. (canceled)

7. The fusion protein of claim 1, wherein the fusion protein consists of the structure
(B-A-C)_{y}.

8. The fusion protein of claim 1, wherein B comprises a chromophore or fluorophore and C comprises a substance that absorbs emission energy of the chromophore or fluorophore wherein B is preferably conjugated to the chromophore or fluorophore; wherein C is preferably a peptide conjugated to a substance that absorbs emission energy of the chromophore or fluorophore; and wherein preferably B comprises a fluorophore and the substance that absorbs emission energy of the chromophore is a quencher.

9. (canceled)

10. (canceled)

11. (canceled)

12. The fusion protein of claim 1, wherein when more than one A is present in the fusion protein, each A is the same.

13. The fusion protein of claim 1, wherein when more than one A is present in the fusion protein, two or more A are different, wherein the two or more A are preferably arranged to bind to the same analyte.

14. (canceled)

15. The fusion protein of claim 1, wherein A comprises an antibody, antibody fragment, peptide aptamer, peptide antigen, or peptide antigen fragment, wherein the peptide antigen or peptide antigen fragment preferably comprises an antigenic determinant or epitope.

16. (canceled)

17. The fusion protein of claim 1, wherein x is 1 or 2.

18. (canceled)

19. (canceled)

20. (canceled)

21. (canceled)

22. The fusion protein of claim 1, wherein B is beta lactamase TEM1 or homologs, fragments and variants thereof, wherein the homologs, fragments and variants at least partially retain enzymatic activity, wherein the beta lactamase TEM1 preferably comprises the amino acid sequence set forth in SEQ ID NO: 1 or homologs, fragments or variants thereof.

23. (canceled)

24. The fusion protein of claim 1, wherein C is beta lactamase inhibitor protein (BLIP) or fragments, homologs and variants thereof that retain at least partially the binding activity for beta lactamase, wherein the BLIP preferably has the amino acid sequence set forth in SEQ ID NO: 2 or homologs, fragments or variants thereof.

25. (canceled)

26. The fusion protein of claim 1, wherein A is an antigenic peptide comprising or consisting of an amino acid sequence selected from the group consisting of those set forth in SEQ ID NOs: 3, 8-10, 12-13.

27. The fusion protein of claim 1, wherein “-” represents a linker comprising more than one amino acids and wherein the linker peptide forms a coiled coil structure.

28. The fusion protein of claim 1, wherein the fusion protein has the amino acid sequence set forth in any one of SEQ ID NOs: 4-5, 11, 14-15.

29. A method for detecting a presence or amount of an analyte molecule, the method comprising:
contacting a fusion protein comprising a structure of
(Aₙ-B-Aₓ-C-Aₘ)_{y}
wherein
each A independently comprises a peptide or protein domain that binds an analyte of interest;
B comprises an entity that can produce a detectable signal;
C comprises an entity that, when A is not bound to the analyte of interest, binds to B and modulates the signal production by B;
n and m are each independently 0 or at least 1;
x is an integer of 0 or at least 1;
y is an integer of at least 1;
“-” represents a covalent bond or a linker comprising or consisting of one or more amino acids;
provided that if x is 0, n, m or both are at least 1, with the analyte molecule under conditions that allow binding of A to the analyte molecule; and
detecting the presence of the analyte molecule by determining a signal produced by the fusion protein:analyte complex.

30. (canceled)

31. (canceled)

32. The method of claim 29, wherein the presence or amount of the analyte molecule is determined by fluorescence, absorbance, luminescence, enzymatic activity, a detectable functional product, a selectable phenotype, a screenable phenotype that produces an activity due to a phenotypic change, wherein the selectable phenotype preferably comprises an antibiotic resistance.

33. (canceled)

34. The method of claim 29, wherein the method is performed in a living cell, wherein the cell is preferably used for a drug screening assay.

35. (canceled)

36. A kit for detecting a presence of an analyte molecule or an amount of analyte, the kit comprising a fusion protein comprising a structure of
(Aₙ-B-Aₓ-C-Aₘ)_{y}
wherein
A independently comprises a peptide or protein domain that binds an analyte of interest;
B comprises an entity that can produce a detectable signal;
C comprises an entity that, when A is not bound to the analyte of interest, binds to B and modulates the signal production by B;
n and m are each independently 0 or at least 1;
x is an integer of 0 or at least 1;
v is an integer of at least 1;
“-” represents a covalent bond or a linker comprising or consisting of one or more amino acids;
provided that if x is 0, n, m or both are at least 1.

37. The kit of claim 36, further comprising a substance for detecting the signal produced by the fusion protein.


SEQUENCE LISTING (15 sequences)

SEQ ID NO: 1 - protein, 286 aa, artificial [sequence omitted]

SEQ ID NO: 2 - protein, 1743 aa, artificial
  Beta lactamase inhibitor protein (BLIP) D49A
MKQNKLLQRGAYFNDKNILIDDFDKRYNDYDFVEFFTGISNSTFGLKSDGNLYACGDNTGFQLGLGKDSSERRMFSKVKIDNVKYVSCGSKHSVAVTKDGFAYGAGTSNVGQLGVIESTVYYEFTKLPIDDVKTVACGYDFTFVLKNDGTLYSAGLNSSGQLGLGDTNNRVTFTKVNIDSVKDVVTYNQSVFIIKMDGTAHACGLNSNGQLGINSTLNKSVFNKIEGMDNVKQIACGSSHTILIKNDGTMYTTGSNGYGQLGTGNNNNSIVFTLSSINNVKYASCGNNHTMILKYDNTLFSTGQNNYGQLANANKDVASRNTFVKVNVENIKDIKCGSQFNFLINGSKEIFVSGCNLAGQLGSFFHTTFLYEFSKVQSSNLDNYSGLLVNDDYLYVTKDNSEFLNVKLSDNFQDYKKIELTDNNMFIVMNDGTLYACGLNNYGQLGLGDTVNRSVMTKVDIDNVLDIKGNGNSTFVLKNNGTLYSCGYNSSGILGLKDNTNRNIFTKIEIENIKEFCVESNYIVALNHSKELYGWGNQSYIVYGDNRNYPYKDTRVSNVEKIATWSDTLYILDSTGATKTIGYSYNGSGGYPAPSSSSTYREGGYINKNTSYRTLEFYNTSKTKLVNLFAFYNGCVFVDENGLAYCIGENNINFRGGSTTNENNSLRFINNSGVYYTNTDGTDYTCYQWTYKLIRCSIFDSPQNIIGNSKNILYLSKNNSTFKCTGNCITYGINSQNWYSYFSDSSNGAIALGNEFILKNYSGECLLKGYGKATNGEFGNSTNISSISNYDTGLKDIKDIIVKNNTVVVVDKNNNIYVTGANQFNKLGIGEYNNQPIRKFTNITEQSNSFIFMDDIKEITTSRNTMFIVKNDGTAYATGNNSSGQLGLGDTINRNKFTQINLDNIKKISTSIDGNTTFAIRNDGTLYSTGLNTKGQLGLGDIVNRNTFTKVNIQNVRDVVLGTTHSHAIKDDNTLYSCGENTHGQLGLGSESNHPDVLTFTVNNITNVRDVYCSDTTTFIVKDTNIAYCCGYNNNSQLGMGNTTDQYSFIKCMENVKEVIPNEINTYIITIYNTAYSTGLNTDYCLGLNSNSNQSSFSEIPISNVVKVAPNRNNAVLLLTSEGDVYTAGKCSNGSGTGSETPEKIKKIASKAKDIGMNYRCGHYVSDNGDLYGTGFNDCGQLGVGNVTKRDTFIKTNTRVKKILPLEYANIAIKDTNDIYICGLNNYGQLGVGNRYDSRNNDNRIFNYKHMNFVMGDLTSIKNRHNFILLNNKIVIPTTKDIDYGLVLGNLYKGDLYTELPYEDIKEVSISKTHIIILLNDGTMYGCGTNYHGELLQDLSINQVDEFVQINVSDVKHVSCGDNFTYFIKSDDSLWSIGKNSEYQLGIGHNNPVTELQRITTISSCKEVHCGKNYTLVVTTSNELFVQGYNDKGALGLGSDSENTIIKFFTKALTDIREIKSYGSDHILVLKNDNSVWVTGKNRDVYKIEQPVEFLKEFTIVPISEDVNTVKDVLATDNTLYIISEVGTTNAAIEITEKSISSIKIKIQDPNKDISRIEMLINGESVKSVSDLITEKISFEVPPDKIKIGENKILFRAYCKGDDLYASLFIFKESTGNSIIKDSYVMIGNRMYKVVNTTSNEQDITITLDRGLEEDLNLGDPIYQLINKTKVQVKINKSDLFKDMKLVEIKKSDSSYQEIYELEEANIKSAQPKIIVEKGDKWTAIKRPSMIFRYDAENNEPQA

SEQ ID NO: 3 - protein, 14 aa, Homo sapiens
GGSEQKLISEEDGG

SEQ ID NO: 4 - protein, 2067 aa, artificial
  BLIP- GGSEQKLISEEDGG-Tem1 (BLIP-c myc-TEM1)
MKQNKLLQRGAYFNDKNILIDDFDKRYNDYDFVEFFTGISNSTFGLKSDGNLYACGDNTGFQLGLGKDSSERRMFSKVKIDNVKYVSCGSKHSVAVTKDGFAYGAGTSNVGQLGVIESTVYYEFTKLPIDDVKTVACGYDFTFVLKNDGTLYSAGLNSSGQLGLGDTNNRVTFTKVNIDSVKDVVTYNQSVFIIKMDGTAHACGLNSNGQLGINSTLNKSVFNKIEGMDNVKQIACGSSHTILIKNDGTMYTTGSNGYGQLGTGNNNNSIVFTLSSINNVKYASCGNNHTMILKYDNTLFSTGQNNYGQLANANKDVASRNTFVKVNVENIKDIKCGSQFNFLINGSKEIFVSGCNLAGQLGSFFHTTFLYEFSKVQSSNLDNYSGLLVNDDYLYVTKDNSEFLNVKLSDNFQDYKKIELTDNNMFIVMNDGTLYACGLNNYGQLGLGDTVNRSVMTKVDIDNVLDIKGNGNSTFVLKNNGTLYSCGYNSSGILGLKDNTNRNIFTKIEIENIKEFCVESNYIVALNHSKELYGWGNQSYIVYGDNRNYPYKDTRVSNVEKIATWSDTLYILDSTGATKTIGYSYNGSGGYPAPSSSSTYREGGYINKNTSYRTLEFYNTSKTKLVNLFAFYNGCVFVDENGLAYCIGENNINFRGGSTTNENNSLRFINNSGVYYTNTDGTDYTCYQWTYKLIRCSIFDSPQNIIGNSKNILYLSKNNSTFKCTGNCITYGINSQNWYSYFSDSSNGAIALGNEFILKNYSGECLLKGYGKATNGEFGNSTNISSISNYDTGLKDIKDIIVKNNTVVVVDKNNNIYVTGANQFNKLGIGEYNNQPIRKFTNITEQSNSFIFMDDIKEITTSRNTMFIVKNDGTAYATGNNSSGQLGLGDTINRNKFTQINLDNIKKISTSIDGNTTFAIRNDGTLYSTGLNTKGQLGLGDIVNRNTFTKVNIQNVRDVVLGTTHSHAIKDDNTLYSCGENTHGQLGLGSESNHPDVLTFTVNNITNVRDVYCSDTTTFIVKDTNIAYCCGYNNNSQLGMGNTTDQYSFIKCMENVKEVIPNEINTYIITIYNTAYSTGLNTDYCLGLNSNSNQSSFSEIPISNVVKVAPNRNNAVLLLTSEGDVYTAGKCSNGSGTGSETPEKIKKIASKAKDIGMNYRCGHYVSDNGDLYGTGFNDCGQLGVGNVTKRDTFIKTNTRVKKILPLEYANIAIKDTNDIYICGLNNYGQLGVGNRYDSRNNDNRIFNYKHMNFVMGDLTSIKNRHNFILLNNKIVIPTTKDIDYGLVLGNLYKGDLYTELPYEDIKEVSISKTHIIILLNDGTMYGCGTNYHGELLQDLSINQVDEFVQINVSDVKHVSCGDNFTYFIKSDDSLWSIGKNSEYQLGIGHNNPVTELQRITTISSCKEVHCGKNYTLVVTTSNELFVQGYNDKGALGLGSDSENTIIKFFTKALTDIREIKSYGSDHILVLKNDNSVWVTGKNRDVYKIEQPVEFLKEFTIVPISEDVNTVKDVLATDNTLYIISEVGTTNAAIEITEKSISSIKIKIQDPNKDISRIEMLINGESVKSVSDLITEKISFEVPPDKIKIGENKILFRAYCKGDDLYASLFIFKESTGNSIIKDSYVMIGNRMYKVVNTTSNEQDITITLDRGLEEDLNLGDPIYQLINKTKVQVKINKSDLFKDMKLVEIKKSDSSYQEIYELEEANIKSAQPKIIVEKGDKWTAIKRPSMIFRYDAENNEPQAGGGSGGSEQKLISEEDGGGGGGSGGGGSGGGGSGGGGGMSIQHFRVALIPFFAAFCLPVFAHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHW

SEQ ID NO: 5 - protein, 2067 aa, artificial
  TEM1- GGSEQKLISEEDGG-BLIP (TEM1-c myc-BLIP)
MSIQHFRVALIPFFAAFCLPVFAHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWGGGSGGSEQKLISEEDGGGGGGSGGGGSGGGGSGGGGGMKQNKLLQRGAYFNDKNILIDDFDKRYNDYDFVEFFTGISNSTFGLKSDGNLYACGDNTGFQLGLGKDSSERRMFSKVKIDNVKYVSCGSKHSVAVTKDGFAYGAGTSNVGQLGVIESTVYYEFTKLPIDDVKTVACGYDFTFVLKNDGTLYSAGLNSSGQLGLGDTNNRVTFTKVNIDSVKDVVTYNQSVFIIKMDGTAHACGLNSNGQLGINSTLNKSVFNKIEGMDNVKQIACGSSHTILIKNDGTMYTTGSNGYGQLGTGNNNNSIVFTLSSINNVKYASCGNNHTMILKYDNTLFSTGQNNYGQLANANKDVASRNTFVKVNVENIKDIKCGSQFNFLINGSKEIFVSGCNLAGQLGSFFHTTFLYEFSKVQSSNLDNYSGLLVNDDYLYVTKDNSEFLNVKLSDNFQDYKKIELTDNNMFIVMNDGTLYACGLNNYGQLGLGDTVNRSVMTKVDIDNVLDIKGNGNSTFVLKNNGTLYSCGYNSSGILGLKDNTNRNIFTKIEIENIKEFCVESNYIVALNHSKELYGWGNQSYIVYGDNRNYPYKDTRVSNVEKIATWSDTLYILDSTGATKTIGYSYNGSGGYPAPSSSSTYREGGYINKNTSYRTLEFYNTSKTKLVNLFAFYNGCVFVDENGLAYCIGENNINFRGGSTTNENNSLRFINNSGVYYTNTDGTDYTCYQWTYKLIRCSIFDSPQNIIGNSKNILYLSKNNSTFKCTGNCITYGINSQNWYSYFSDSSNGAIALGNEFILKNYSGECLLKGYGKATNGEFGNSTNISSISNYDTGLKDIKDIIVKNNTVVVVDKNNNIYVTGANQFNKLGIGEYNNQPIRKFTNITEQSNSFIFMDDIKEITTSRNTMFIVKNDGTAYATGNNSSGQLGLGDTINRNKFTQINLDNIKKISTSIDGNTTFAIRNDGTLYSTGLNTKGQLGLGDIVNRNTFTKVNIQNVRDVVLGTTHSHAIKDDNTLYSCGENTHGQLGLGSESNHPDVLTFTVNNITNVRDVYCSDTTTFIVKDTNIAYCCGYNNNSQLGMGNTTDQYSFIKCMENVKEVIPNEINTYIITIYNTAYSTGLNTDYCLGLNSNSNQSSFSEIPISNVVKVAPNRNNAVLLLTSEGDVYTAGKCSNGSGTGSETPEKIKKIASKAKDIGMNYRCGHYVSDNGDLYGTGFNDCGQLGVGNVTKRDTFIKTNTRVKKILPLEYANIAIKDTNDIYICGLNNYGQLGVGNRYDSRNNDNRIFNYKHMNFVMGDLTSIKNRHNFILLNNKIVIPTTKDIDYGLVLGNLYKGDLYTELPYEDIKEVSISKTHIIILLNDGTMYGCGTNYHGELLQDLSINQVDEFVQINVSDVKHVSCGDNFTYFIKSDDSLWSIGKNSEYQLGIGHNNPVTELQRITTISSCKEVHCGKNYTLVVTTSNELFVQGYNDKGALGLGSDSENTIIKFFTKALTDIREIKSYGSDHILVLKNDNSVWVTGKNRDVYKIEQPVEFLKEFTIVPISEDVNTVKDVLATDNTLYIISEVGTTNAAIEITEKSISSIKIKIQDPNKDISRIEMLINGESVKSVSDLITEKISFEVPPDKIKIGENKILFRAYCKGDDLYASLFIFKESTGNSIIKDSYVMIGNRMYKVVNTTSNEQDITITLDRGLEEDLNLGDPIYQLINKTKVQVKINKSDLFKDMKLVEIKKSDSSYQEIYELEEANIKSAQPKIIVEKGDKWTAIKRPSMIFRYDAENNEPQA

SEQ ID NO: 6 - protein, 4 aa, artificial
  Linker
GGGS

SEQ ID NO: 7 - protein, 20 aa, artificial
  Linker
GGGGSGGGGSGGGGSGGGGG

SEQ ID NO: 8 - protein, 6 aa, Human immunodeficiency virus
EKIRLR

SEQ ID NO: 9 - protein, 7 aa, Homo sapiens
SDLWKLL

SEQ ID NO: 10 - protein, 491 aa, Homo sapiens
MCNTNMSVPTDGAVTTSQIPASEQETLVRPKPLLLKLLKSVGAQKDTYTMKEVLFYLGQYIMTKRLYDEKQQHIVYCSNDLLGDLFGVPSFSVKEHRKIYTMIYRNLVVVNQQESSDSGTSVSENRCHLEGGSDQKDLVQELQEEKPSSSHLVSRPSTSSRRRAISETEENSDELSGERQRKRHKSDSISLSFDESLALCVIREICCERSSSSESTGTPSNPDLDAGVSEHSGDWLDQDSVSDQFSVEFEVESLDSEDYSLSEEGQELSDEDDEVYQVTVYQAGESDTDSFEEDPEISLADYWKCTSCNEMNPPLPSHCNRCWALRENWLPEDKGKDKGEISEKAKLENSTQAEEGFDVPDCKKTIVNDSRESCVEENDDKITQASQSQESEDYSQPSTSSSIIYSSQEDVKEFEREETQDKEESVESSLPLNAIEPCVICQGRPKNGCIVHGKTGHLMACFTCAKKLKKRNKPCPVCRQPIQMIVLTYFP

SEQ ID NO: 11 - protein, 2060 aa, artificial
  Tem1-SDLWKLL-BLIP
MSIQHFRVALIPFFAAFCLPVFAHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWGGGSSDLWKLLGGGGSGGGGSGGGGSGGGGGMKQNKLLQRGAYFNDKNILIDDFDKRYNDYDFVEFFTGISNSTFGLKSDGNLYACGDNTGFQLGLGKDSSERRMFSKVKIDNVKYVSCGSKHSVAVTKDGFAYGAGTSNVGQLGVIESTVYYEFTKLPIDDVKTVACGYDFTFVLKNDGTLYSAGLNSSGQLGLGDTNNRVTFTKVNIDSVKDVVTYNQSVFIIKMDGTAHACGLNSNGQLGINSTLNKSVFNKIEGMDNVKQIACGSSHTILIKNDGTMYTTGSNGYGQLGTGNNNNSIVFTLSSINNVKYASCGNNHTMILKYDNTLFSTGQNNYGQLANANKDVASRNTFVKVNVENIKDIKCGSQFNFLINGSKEIFVSGCNLAGQLGSFFHTTFLYEFSKVQSSNLDNYSGLLVNDDYLYVTKDNSEFLNVKLSDNFQDYKKIELTDNNMFIVMNDGTLYACGLNNYGQLGLGDTVNRSVMTKVDIDNVLDIKGNGNSTFVLKNNGTLYSCGYNSSGILGLKDNTNRNIFTKIEIENIKEFCVESNYIVALNHSKELYGWGNQSYIVYGDNRNYPYKDTRVSNVEKIATWSDTLYILDSTGATKTIGYSYNGSGGYPAPSSSSTYREGGYINKNTSYRTLEFYNTSKTKLVNLFAFYNGCVFVDENGLAYCIGENNINFRGGSTTNENNSLRFINNSGVYYTNTDGTDYTCYQWTYKLIRCSIFDSPQNIIGNSKNILYLSKNNSTFKCTGNCITYGINSQNWYSYFSDSSNGAIALGNEFILKNYSGECLLKGYGKATNGEFGNSTNISSISNYDTGLKDIKDIIVKNNTVVVVDKNNNIYVTGANQFNKLGIGEYNNQPIRKFTNITEQSNSFIFMDDIKEITTSRNTMFIVKNDGTAYATGNNSSGQLGLGDTINRNKFTQINLDNIKKISTSIDGNTTFAIRNDGTLYSTGLNTKGQLGLGDIVNRNTFTKVNIQNVRDVVLGTTHSHAIKDDNTLYSCGENTHGQLGLGSESNHPDVLTFTVNNITNVRDVYCSDTTTFIVKDTNIAYCCGYNNNSQLGMGNTTDQYSFIKCMENVKEVIPNEINTYIITIYNTAYSTGLNTDYCLGLNSNSNQSSFSEIPISNVVKVAPNRNNAVLLLTSEGDVYTAGKCSNGSGTGSETPEKIKKIASKAKDIGMNYRCGHYVSDNGDLYGTGFNDCGQLGVGNVTKRDTFIKTNTRVKKILPLEYANIAIKDTNDIYICGLNNYGQLGVGNRYDSRNNDNRIFNYKHMNFVMGDLTSIKNRHNFILLNNKIVIPTTKDIDYGLVLGNLYKGDLYTELPYEDIKEVSISKTHIIILLNDGTMYGCGTNYHGELLQDLSINQVDEFVQINVSDVKHVSCGDNFTYFIKSDDSLWSIGKNSEYQLGIGHNNPVTELQRITTISSCKEVHCGKNYTLVVTTSNELFVQGYNDKGALGLGSDSENTIIKFFTKALTDIREIKSYGSDHILVLKNDNSVWVTGKNRDVYKIEQPVEFLKEFTIVPISEDVNTVKDVLATDNTLYIISEVGTTNAAIEITEKSISSIKIKIQDPNKDISRIEMLINGESVKSVSDLITEKISFEVPPDKIKIGENKILFRAYCKGDDLYASLFIFKESTGNSIIKDSYVMIGNRMYKVVNTTSNEQDITITLDRGLEEDLNLGDPIYQLINKTKVQVKINKSDLFKDMKLVEIKKSDSSYQEIYELEEANIKSAQPKIIVEKGDKWTAIKRPSMIFRYDAENNEPQA

SEQ ID NO: 12 - protein, 7 aa, Homo sapiens
SGLWKLL

SEQ ID NO: 13 - protein, 7 aa, Homo sapiens
AGLWKLL

SEQ ID NO: 14 - protein, 2060 aa, artificial
  TEM1-SGLWKLL-BLIP
MSIQHFRVALIPFFAAFCLPVFAHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWGGGSSGLWKLLGGGGSGGGGSGGGGSGGGGGMKQNKLLQRGAYFNDKNILIDDFDKRYNDYDFVEFFTGISNSTFGLKSDGNLYACGDNTGFQLGLGKDSSERRMFSKVKIDNVKYVSCGSKHSVAVTKDGFAYGAGTSNVGQLGVIESTVYYEFTKLPIDDVKTVACGYDFTFVLKNDGTLYSAGLNSSGQLGLGDTNNRVTFTKVNIDSVKDVVTYNQSVFIIKMDGTAHACGLNSNGQLGINSTLNKSVFNKIEGMDNVKQIACGSSHTILIKNDGTMYTTGSNGYGQLGTGNNNNSIVFTLSSINNVKYASCGNNHTMILKYDNTLFSTGQNNYGQLANANKDVASRNTFVKVNVENIKDIKCGSQFNFLINGSKEIFVSGCNLAGQLGSFFHTTFLYEFSKVQSSNLDNYSGLLVNDDYLYVTKDNSEFLNVKLSDNFQDYKKIELTDNNMFIVMNDGTLYACGLNNYGQLGLGDTVNRSVMTKVDIDNVLDIKGNGNSTFVLKNNGTLYSCGYNSSGILGLKDNTNRNIFTKIEIENIKEFCVESNYIVALNHSKELYGWGNQSYIVYGDNRNYPYKDTRVSNVEKIATWSDTLYILDSTGATKTIGYSYNGSGGYPAPSSSSTYREGGYINKNTSYRTLEFYNTSKTKLVNLFAFYNGCVFVDENGLAYCIGENNINFRGGSTTNENNSLRFINNSGVYYTNTDGTDYTCYQWTYKLIRCSIFDSPQNIIGNSKNILYLSKNNSTFKCTGNCITYGINSQNWYSYFSDSSNGAIALGNEFILKNYSGECLLKGYGKATNGEFGNSTNISSISNYDTGLKDIKDIIVKNNTVVVVDKNNNIYVTGANQFNKLGIGEYNNQPIRKFTNITEQSNSFIFMDDIKEITTSRNTMFIVKNDGTAYATGNNSSGQLGLGDTINRNKFTQINLDNIKKISTSIDGNTTFAIRNDGTLYSTGLNTKGQLGLGDIVNRNTFTKVNIQNVRDVVLGTTHSHAIKDDNTLYSCGENTHGQLGLGSESNHPDVLTFTVNNITNVRDVYCSDTTTFIVKDTNIAYCCGYNNNSQLGMGNTTDQYSFIKCMENVKEVIPNEINTYIITIYNTAYSTGLNTDYCLGLNSNSNQSSFSEIPISNVVKVAPNRNNAVLLLTSEGDVYTAGKCSNGSGTGSETPEKIKKIASKAKDIGMNYRCGHYVSDNGDLYGTGFNDCGQLGVGNVTKRDTFIKTNTRVKKILPLEYANIAIKDTNDIYICGLNNYGQLGVGNRYDSRNNDNRIFNYKHMNFVMGDLTSIKNRHNFILLNNKIVIPTTKDIDYGLVLGNLYKGDLYTELPYEDIKEVSISKTHIIILLNDGTMYGCGTNYHGELLQDLSINQVDEFVQINVSDVKHVSCGDNFTYFIKSDDSLWSIGKNSEYQLGIGHNNPVTELQRITTISSCKEVHCGKNYTLVVTTSNELFVQGYNDKGALGLGSDSENTIIKFFTKALTDIREIKSYGSDHILVLKNDNSVWVTGKNRDVYKIEQPVEFLKEFTIVPISEDVNTVKDVLATDNTLYIISEVGTTNAAIEITEKSISSIKIKIQDPNKDISRIEMLINGESVKSVSDLITEKISFEVPPDKIKIGENKILFRAYCKGDDLYASLFIFKESTGNSIIKDSYVMIGNRMYKVVNTTSNEQDITITLDRGLEEDLNLGDPIYQLINKTKVQVKINKSDLFKDMKLVEIKKSDSSYQEIYELEEANIKSAQPKIIVEKGDKWTAIKRPSMIFRYDAENNEPQA

SEQ ID NO: 15 - protein, 2060 aa, artificial
  TEM1-AGLWKLL-BLIP
MSIQHFRVALIPFFAAFCLPVFAHPETLVKVKDAEDQLGARVGYIELDLNSGKILESFRPEERFPMMSTFKVLLCGAVLSRVDAGQEQLGRRIHYSQNDLVEYSPVTEKHLTDGMTVRELCSAAITMSDNTAANLLLTTIGGPKELTAFLHNMGDHVTRLDRWEPELNEAIPNDERDTTMPAAMATTLRKLLTGELLTLASRQQLIDWMEADKVAGPLLRSALPAGWFIADKSGAGERGSRGIIAALGPDGKPSRIVVIYTTGSQATMDERNRQIAEIGASLIKHWGGGSAGLWKLLGGGGSGGGGSGGGGSGGGGGMKQNKLLQRGAYFNDKNILIDDFDKRYNDYDFVEFFTGISNSTFGLKSDGNLYACGDNTGFQLGLGKDSSERRMFSKVKIDNVKYVSCGSKHSVAVTKDGFAYGAGTSNVGQLGVIESTVYYEFTKLPIDDVKTVACGYDFTFVLKNDGTLYSAGLNSSGQLGLGDTNNRVTFTKVNIDSVKDVVTYNQSVFIIKMDGTAHACGLNSNGQLGINSTLNKSVFNKIEGMDNVKQIACGSSHTILIKNDGTMYTTGSNGYGQLGTGNNNNSIVFTLSSINNVKYASCGNNHTMILKYDNTLFSTGQNNYGQLANANKDVASRNTFVKVNVENIKDIKCGSQFNFLINGSKEIFVSGCNLAGQLGSFFHTTFLYEFSKVQSSNLDNYSGLLVNDDYLYVTKDNSEFLNVKLSDNFQDYKKIELTDNNMFIVMNDGTLYACGLNNYGQLGLGDTVNRSVMTKVDIDNVLDIKGNGNSTFVLKNNGTLYSCGYNSSGILGLKDNTNRNIFTKIEIENIKEFCVESNYIVALNHSKELYGWGNQSYIVYGDNRNYPYKDTRVSNVEKIATWSDTLYILDSTGATKTIGYSYNGSGGYPAPSSSSTYREGGYINKNTSYRTLEFYNTSKTKLVNLFAFYNGCVFVDENGLAYCIGENNINFRGGSTTNENNSLRFINNSGVYYTNTDGTDYTCYQWTYKLIRCSIFDSPQNIIGNSKNILYLSKNNSTFKCTGNCITYGINSQNWYSYFSDSSNGAIALGNEFILKNYSGECLLKGYGKATNGEFGNSTNISSISNYDTGLKDIKDIIVKNNTVVVVDKNNNIYVTGANQFNKLGIGEYNNQPIRKFTNITEQSNSFIFMDDIKEITTSRNTMFIVKNDGTAYATGNNSSGQLGLGDTINRNKFTQINLDNIKKISTSIDGNTTFAIRNDGTLYSTGLNTKGQLGLGDIVNRNTFTKVNIQNVRDVVLGTTHSHAIKDDNTLYSCGENTHGQLGLGSESNHPDVLTFTVNNITNVRDVYCSDTTTFIVKDTNIAYCCGYNNNSQLGMGNTTDQYSFIKCMENVKEVIPNEINTYIITIYNTAYSTGLNTDYCLGLNSNSNQSSFSEIPISNVVKVAPNRNNAVLLLTSEGDVYTAGKCSNGSGTGSETPEKIKKIASKAKDIGMNYRCGHYVSDNGDLYGTGFNDCGQLGVGNVTKRDTFIKTNTRVKKILPLEYANIAIKDTNDIYICGLNNYGQLGVGNRYDSRNNDNRIFNYKHMNFVMGDLTSIKNRHNFILLNNKIVIPTTKDIDYGLVLGNLYKGDLYTELPYEDIKEVSISKTHIIILLNDGTMYGCGTNYHGELLQDLSINQVDEFVQINVSDVKHVSCGDNFTYFIKSDDSLWSIGKNSEYQLGIGHNNPVTELQRITTISSCKEVHCGKNYTLVVTTSNELFVQGYNDKGALGLGSDSENTIIKFFTKALTDIREIKSYGSDHILVLKNDNSVWVTGKNRDVYKIEQPVEFLKEFTIVPISEDVNTVKDVLATDNTLYIISEVGTTNAAIEITEKSISSIKIKIQDPNKDISRIEMLINGESVKSVSDLITEKISFEVPPDKIKIGENKILFRAYCKGDDLYASLFIFKESTGNSIIKDSYVMIGNRMYKVVNTTSNEQDITITLDRGLEEDLNLGDPIYQLINKTKVQVKINKSDLFKDMKLVEIKKSDSSYQEIYELEEANIKSAQPKIIVEKGDKWTAIKRPSMIFRYDAENNEPQA